(19) United States
(12) Patent Application Publication
(10) Pub. No.: US 2002/0177132 A1
(43) Pub. Date: Nov. 28, 2002

(54) Method and system for the analysis of variance of microarray data

(76) Inventors: M. Kathleen Kerr, Bar Harbor, ME (US); Gary A. Churchill, Bar Harbor, ME (US)

Correspondence Address: PENNIE AND EDMONDS, 1155 AVENUE OF THE AMERICAS, NEW YORK, NY, 100362711

(21) Appl. No.: 09/865,627

(22) Filed: May 25, 2001

(51) Int. Cl. C12Q 1/68; G06F 19/00; G01N 33/48; G01N 33/50

(52) U.S. Cl. 435/6; 702/20

(57) ABSTRACT

A method for estimating the factor and interaction effects for gene expression microarray experiments is disclosed. The method requires the inversion of two square matrices of size p and p′, respectively, instead of a matrix of size q where q>>p≈p′. The invention also includes implementation of the methods in computer software, computer readable media comprising these software instructions, and computer systems for performing the methods.

### FIELD OF INVENTION

This invention relates to an efficient method for the analysis of high dimensionality datasets. Specifically, the invention relates to the analysis of variance of DNA microarray data, for example, from high-throughput gene expression experiments.

### BACKGROUND OF THE INVENTION

Citation or identification of any reference in this or any of the sections of this application shall not be construed to mean that it is available as prior art to the present invention.

DNA microarray experiments are powerful and cost-effective ways for determining gene expression with many applications. Such experiments have been used to study gene expression in yeast under different stress condition, gene expression profiles for tumors from cancer patients, and gene expression in the livers of mice representing a model of maturity-onset type II diabetes wherein one group of mice is fed a beta-3 adrenergic receptor agonist. In addition to helping scientists understand gene regulation and interactions, microarray experiments may be used to identify disease genes and targets for therapeutic drugs.

In a typical DNA microarray experiment, a microarray is prepared by fixing or synthesizing known polynucleotides to a suitable substrate in a grid pattern. Each spot in the microarray is comprised of a purified polynucleotide and each polynucleotide may be placed in several spots on the microarray. Each spot is referred to herein as a “probe” or “gene.” As used herein, “probe” and “target” follow the definitions adopted in The Chipping Forecast, volume 21, Supplement to Nature Genetics, 1999. The microarray may have thousands of polynucleotide spots contained in an area of about 2 cm on each side. The microarrays are usually produced by an automated mechanical printing process so that the same polynucleotide is spotted on the same location in each array. Alternatively, the microarray may be produced by synthesizing the polynucleotides directly on the surface of the substrate.

Pools of purified mRNA are prepared from cell populations under study and reverse-transcribed into cDNA. The cDNA samples are labeled with fluorescent dyes such as the red fluorescent dye, Cy5, and the green fluorescent dye, Cy3. Other dyes may be used to tag the cDNA targets so the tagged targets are usually referred to by the Cy5/Cy3 colors, “red” and “green.” The labeled cDNA samples are referred to herein as the “target” or “variety.” The purpose of a typical cDNA microarray experiment is to determine the effect, if any, between the genes on the microarray and the labeled cDNA varieties.

In one type of cDNA microarray experiment, two varieties are used: one taken from a diseased cell line and one taken from a healthy cell line. The goal of such an experiment is to identify significant differences between the expression of particular genes in the healthy and diseased states.

In another type of cDNA microarray experiment, the number of varieties examined may be as high as a hundred or more wherein samples from a single cell line undergoing a process being investigated are taken at various times during the process. Each sample taken at a particular time represents a variety and the number of varieties in the experiment equals the number of samples taken.

One-half of each variety is labeled with the red dye and the other half is labeled with the green dye. If the experiment involves only two varieties, the red half of the first variety is mixed with the green half of the second variety to form a first test sample. In a preferred embodiment, the green half of the first variety is mixed with the red half of the second variety to form a second test sample. The first test sample is applied to a prepared microarray and the microarray is incubated for a set period and temperature wherein the cDNA of the first test sample is allowed to competitively hybridize with the genes printed on the microarray. After incubation, the microarray is washed to remove the unhybridized cDNA. The washed microarray is illuminated by light that causes the red and green tags to emit fluorescent light. The microarray is scanned wherein the intensities of the fluorescent light emitted by the red and green dyes are measured and recorded for each spot on the microarray. The intensities of the fluorescent dye signals depend, in part, on the abundance of the corresponding mRNA in the sample.

In a preferred embodiment, the second test sample is applied to a second microarray prepared identically to the first microarray and allowed to competitively hybridize with the genes printed on the second microarray. After incubation, the second microarray is washed to remove the unhybridized cDNA and illuminated by light causing the red and green tags to emit fluorescent light. The second microarray is scanned and the intensities of the fluorescent light emitted by the red and green dyes are measured and recorded for each spot on the second microarray.

If the experiment has more than two varieties, the tagged halves of each variety may be combined with other varieties in a loop design as described in Kerr et al., “Experimental Design for Gene Expression Microarrays”, [online], (2000) The Jackson Laboratory, [retrieved on 2001-05-01], retrieved from the Internet:<URL: http://www.jax.org/research/churchill/research/expression/kerr-design.pdf> herein incorporated by reference in its entirety. In a loop design, each variety is labeled once with the red and green dye. In such a design, the varieties are said to be balanced with respect to dyes and means that dye effects are unconfounded with variety effects. Each solution is applied to an identically prepared microarray. The microarrays are then incubated, washed, and scanned as described for the two- variety experiment.

Early experiments with microarrays calculated the ratio between the red and green dye intensities for each spot on the microarray. If both targets hybridized to the probe at equal rates, the red and green intensities for the spot would be roughly equal and the R/G ratio would be about one. If, on the other hand, the red target hybridized at a much higher rate than the green target to the probe, the measured red intensity signal would be larger than the green intensity signal and the R/G ratio would be larger than one. Conversely, if the green target hybridized at a higher rate than the red target to the probe, the R/G ratio would be a small fraction of one. Microarray experiments, however, contain a large amount of variability due to the methods used for preparing and purifying the gene and cDNA samples, spotting the polynucleotides on the microarray, scanning the washed microarray after incubation, and the variability that arises from the inherent complexity of biological systems. The early experiments with microarrays addressed the variability issue by setting an arbitrary threshold level for the intensity ratios. In the early experiments, for example, only ratios exceeding two to three times the average of all the intensity ratios in the experiment were considered significant.

Simple ratios are adequate for identifying genes with large changes in expression but cannot detect small changes in expression. In order to identify genes with small, but reproducible, changes in expression, statistical methods must be used to analyze the microarray data.

Dudoit, et al., “Statistical methods for identifying differentially expressed genes in replicated cDNA microarray experiments”, Technical report #578, Department of Statistics, University of California, Berkeley, [online], 2000 [retrieved on 2001-05-15], retrieved from the Internet:<URL:http://www.stat.berkeley.edu/users/terry/zarray/Html/matt.html> discloses statistical methods for identifying differentially expressed genes using a t-statistic with modified p-values on the log₂(R/G) intensity ratios for each gene in the experiment. The intensity ratios are first normalized to remove the identified systematic variation in the microarray experiments as described in Yang., et al., “Normalization for cDNA Microarray Data”, Technical report #589, Department of Statistics, University of California, Berkeley, [online], 2001 [retrieved on 2001-05-15], retrieved from the Internet:<http://www.stat.berkeley.edu/users/terry/zarray/TechReport/589.pdf>.

Analysis of Variance (ANOVA) is another method of analyzing microarray datasets. ANOVA is generally described in Montgomery, D. C. Design and analysis of experiments, NY, John Wiley & Sons, 1991, pp.1-515, QA279.M66. A description of ANOVA applied to microarray datasets is given in Kerr, et al., “Analysis of Variance for Gene Expression Microarray Data”, Journal of Computational Biology 7, 819-837 (2000), herein incorporated by reference.

ANOVA allocates the variation in an experiment to multiple sources and is capable of discerning smaller effects that the threshold ratio technique cannot handle. One step in the ANOVA procedure requires the calculation of the inverse of a matrix of size q where q is the number of parameters (also referred to as the dimensionality of the problem) in the microarray experiment. In a typical microarray experiment, the matrix size, q, may be several thousand and require significant computational resources in order to determine the inverse matrix. Therefore, there exists a need to efficiently perform the ANOVA by reducing the dimensionality of the matrix.

### SUMMARY OF THE INVENTION

An efficient method for the analysis of variance of gene expression microarray datasets is disclosed for experimental designs wherein the gene factor is orthogonal to the other factors in the experiment (A, D & V). The orthogonality of the G factor to the other factors removes the gene-specific terms from the least-squares normal equations for the non-G factors thereby allowing a sequential solution of first estimating the main effects followed by estimating the gene-specific effects that does not require the inversion of a large matrix of size about n(a+v) where a is the number of microarrays in the experiment, v is the number of varieties in the experiment and n is the number of genes in the experiment. The main effects are first estimated which requires the inversion of a matrix of size about (a+v−1)<<n(a+v). The main effects are then used to estimate two-factor interaction effects for each gene that requires the inverting a matrix of size (a+v−2) only once.

In one embodiment of the present invention, a method for estimating the effects of a plurality of factors and at least one of a plurality of interactions between the factors in a gene expression microarray experiment generating a microarray dataset wherein the factors include a gene factor and a variety factor and the interactions include a variety-gene interaction, the gene factor being orthogonal to the other factors, the method comprising the steps of estimating the factor effects based on a plurality of averages of the microarray dataset and estimating the interaction effects based on a plurality of averages of the microarray dataset and on the estimated factor effects. Furthermore, estimating the factor effects includes inverting a square matrix of size p wherein p is equal to the sum of the number of levels for each non-gene factor minus the number of non-gene factors. In addition, the interaction effects are estimated by inverting a second square matrix of size p′ wherein p′ is p−1.

In another embodiment of the present invention, a method is disclosed for estimating at least one gene-variety interaction in a gene expression microarray experiment having an experimental design characterized by a number of degrees of freedom, q, and defined by a gene factor, a plurality of non-gene factors, a plurality of two-factor interactions wherein a full replication of genes is present for every combination of the plurality of non-gene factors, the method comprising the steps of: inverting a first square matrix characterized by a size, p, wherein p<q; estimating at least one of a plurality of non-gene factor effect from the first square matrix inverse; constructing a second square matrix based in part on the estimated non-gene factor, the second square matrix characterized by size, p′, wherein p′<q; inverting a second square matrix; and estimating at least one gene-variety interaction from the inverted second square matrix.

In another embodiment of the present invention, a method is disclosed for estimating at least one gene-variety interaction in a gene expression microarray experiment generating a dataset and having a design characterized by a arrays, v varieties, n genes, and d dyes wherein a full replication of genes is present for every combination of arrays, varieties and dyes, the method comprising the steps of: constructing a global data vector, d, based on a plurality of averages of the dataset; constructing a square matrix, T, characterized by a size, p, wherein p=a+v+d−3; inverting the square matrix, T; estimating the global effects, τ wherein τ=T d; constructing a square matrix, T_{g}, characterized by a size, p′, wherein p′=p−1; constructing a gene-specific data vector, d_{g}, based on a plurality of averages of the dataset; inverting the square matrix, T_{g}; and estimating the gene-variety interaction, τ_{g}, wherein τ_{g}=T_{g} d_{g}.

In another embodiment of the present invention, a system is disclosed for estimating the effects of a plurality of factors and at least one of a plurality of interactions between the factors in a gene expression microarray experiment generating a microarray dataset wherein the factors include a gene factor and a variety factor and the interactions include a variety-gene interaction, the gene factor being orthogonal to the other factors, the system comprising: a processor; a memory in signal communication with the processor; and a program stored in the memory, the program capable of being executed by the processor, the program including the steps of estimating the main effects based on a plurality of averages of the microarray dataset; and estimating the interaction effects based on a plurality of averages of the microarray dataset and on the estimated factor effects.

In another embodiment of the present invention, a method is disclosed for estimating the effects of a plurality of factors and at least one of a plurality of interactions between the factors in a gene expression microarray experiment generating a microarray dataset wherein the factors include a gene factor and a plurality of non-gene factors and the interactions include at least one of a gene-non-gene interaction, the gene factor being orthogonal to the non-gene factors, the method comprising the steps of: constructing a first data model including only non-gene factors and non-gene interactions; estimating the effects of the non-gene factors and non-gene interactions based on the first data model and on a plurality of averages of the microarray dataset; creating a transformed dataset from the microarray dataset and the estimated factor and interaction effects; constructing a second data model including the gene factors and the gene interactions; and estimating the gene-non-gene interaction effects based on the second data model and a plurality of averages of the transformed dataset.

### BRIEF DESCRIPTION OF THE DRAWINGS

The present invention may be understood more fully by reference to the following detailed description of the preferred embodiment of the present invention, illustrative examples of specific embodiments of the invention and the appended figures in which:

FIG. 1 is a flowchart illustrating the method of a preferred embodiment of the present invention.

FIG. 2 is a block diagram of a preferred embodiment of the present invention.

### DETAILED DESCRIPTION OF THE PREFERRED EMBODIMENTS

A typical microarray experimental design is described below in order to define the variables used in a preferred embodiment of the present invention. It will become apparent to those skilled in the art that the present invention is not limited to the particular design described below.

The goal of many experiments is to determine the effect of one or more independent variables on one or more dependent variables. Independent variables are controlled by the experimenter and the dependent variables are the quantities measured by the experimenter. In a microarray experiment, the single dependent variable is the measured fluorescent light intensities emitted by each dye on each spot in the microarray. The independent variables in the microarray experiment are the probes (genes) and targets (varieties).

In addition to independent variables, “environmental” variables may affect the measured response of the dependent variables to such an extent that the experimenter must consider such environmental variables during both the design of the experiment and during the analysis of the experiment's dataset. One example of such a variable is the variation caused by slight differences between each microarray when more than one microarray is used in an experiment. Although every effort is made to produce identical microarrays, even slight variations in spotting, for example, may result in a response bias that could mask the effects the experimenter is ultimately interested in determining. Another example is the different quantum efficiencies of the dyes used in the experiment. The dye with a higher quantum efficiency will tend to emit more fluorescent light than the dye with the lower quantum efficiency. Since the scanner cannot distinguish the fluorescent light emitted by a “brighter” dye and the fluorescent light emitted by a strongly hybridized target, unless the systematic bias introduced by the dyes is compensated or cancelled, the experimenter will not be able to distinguish the hybridization effects from the dye effects, especially when the two effects are roughly of the same strength.

In a preferred embodiment, the two independent variables, variety (“V”) and gene (“G”), and two environmental variables, array (“A”) and dye (“D”), are selected as the factors of the experiment design. Each factor has a set number of levels. For example, the dye factor, D, has two levels designated “red” and “green” in the preferred embodiment. The number of levels for the array factor, A, is equal to the number of microarrays used in the experiment and is designated by “d”. Similarly, the number of levels for the variety factor, V, is equal to the number of targets used in the experiment and is designated by “v”. The number of levels for the gene factor, G, is equal to the number of distinct probes used in the experiment and is designated by “n”. The effect of each of the factors on the measured response are called the main effects.

In addition to the four main effects, there are six 2-factor interactions, four 3-factor interactions, and one 4-factor interaction. Not all interactions are expected to be significant and the experimenter selects the interactions considered based on experience. In a preferred embodiment, the variety x gene (“VG”) interaction and the array x gene (“AG”) interaction are selected for the data model. The VG interaction accounts for the effect of variety-gene pairs on the measured fluorescent light intensity. A large effect of a specific variety-gene pair indicates gene expression. The AG interaction accounts for the effect of array-gene pairs on the measured fluorescent light intensity.

In order to allocate the variation in the measured dataset to identified sources of variation, a data model is constructed from factors of interest and from known or suspected sources of variation. For purposes of illustration, a four-factor linear data model including the four main effects and two two-factor interactions is chosen having the following form:

y_{ijkgs}=μ+Aᵢ+Dⱼ+Vₖ+G_{g}+(AG)_{igs}+(VG)_{kg}+ε_{ijkgs} (1)

where

y_{ijkgs} is the measured fluorescent light intensity from the s^{th} spot of the i^{th} array, j^{th} dye, k^{th} variety, and g^{th} gene,

μ is the average of all measurements,

Aᵢ is the effect of the i^{th} array,

Dⱼ is the effect of the j^{th} dye,

Vₖ is the effect of the k^{th} variety,

G_{g} is the effect of the g^{th} gene,

(AG)_{igs} is the effect of the interaction between the i^{th} array and the g^{th} gene,

(VG)_{kg} is the effect of the interaction between the kₜₕ variety and the gₜₕ gene, and

ε_{ijkgs} is the mean zero independent error term of the model.

The main effect Aᵢ accounts for the variation that each individual array sees during fabrication and during the experiment that contributes to the variation in the fluorescent signal from array to array. It accounts for variation that may occur when arrays are probed under inconsistent conditions that increase or reduce hybridization efficiencies of the labeled cDNA. The index, i, ranges from 1 to a where a is equal to the number of microarrays used in the experiment. For example, when two microarrays are used in the experiment, a=2 which is also equal to the A-factor levels. The number of degrees of freedom (“dof”) for the A factor is equal to (a−1).

The dye main effect, Dⱼ, measures the differences in the two dye fluorescent labels. An example of such a difference may occur because one dye is consistently “brighter” than the other dye. The index, j, ranges from 1 to d where d is equal to the number of dyes used in the experiment. In a typical gene expression microarray experiment, two dyes are used, red and green, so d=2 and the D-factor levels 2. The dof for the D factor is equal to (d−1).

The variety main effect, Vₖ, accounts for the variation that arises when specific varieties have higher or lower expression levels for all the genes spotted on the arrays. The index, k, ranges from 1 to v where v is equal to the number of varieties used in the experiment. The V-factor levels are also equal to v and the dof for the V factor is equal to (v−1).

The gene main effect, G_{g}, accounts for the variation that arises when certain genes emit a higher or lower fluorescent signal overall compared to other genes. This may occur because some genes have generally higher or lower levels of expression than other genes or may occur because of the different hybridization efficiencies and different labeling efficiencies for the different genes. The index, g, ranges from 1 to n where n is equal to the number of genes used in the experiment. The G-factor levels are also equal to n and the dof for the G factor is equal to (n−1).

The 2-factor array-gene (AG) interaction accounts for the variation between array-gene pairs. The AG interaction, or “spot effects”, arises when the spots for a given gene on the different arrays vary in the amount of cDNA available for hybridization. Since each gene may be replicated on the same microarray, the index, s, ranges from 1 to t where t is equal to the number of times each gene is spotted on the same array. If each gene is spotted only once on each array, s=1.

The 2-factor variety-gene (VG) interaction accounts for the variation between variety-gene pairs and is the information the experiment seeks to resolve.

A complete experiment will involve a arrays, v varieties, and n genes spotted t times on each array. If all possible ijkgs combinations are run, the experiment is called a factorial design. The number of free parameters depends on the data model selected. For the data model described by equation (1), the number of free parameters in the model, q, is (n−1)(v+(a−1)t)+a+v. The number of arrays and varieties are typically less than one hundred but the number of genes may be in the thousands or tens of thousands.

The method of fitting the dataset to the linear model of equation (1) is called linear regression and requires the construction of a design matrix, X, and inverting the q×q matrix X^{T}X. Standard statistical software packages, however, are usually not able to invert the X^{T}X square matrix for the size usually encountered in a cDNA microarray experiment.

The inventors have discovered a novel method that avoids the necessity of inverting a matrix of size q for a certain class of cDNA microarray experimental designs. The microarrays used in the typical cDNA experiment are prepared by a mechanical robot that is programmed to repeatedly print an array in a certain way. Therefore, an assumption may be made that the same set of genes is spotted on each microarray in an experiment. This means that a full replication of genes is present for every array, dye, and variety combination in any experimental design. When such a condition exists, the gene effects are said to be orthogonal to all effects of the array, dye and variety factors. The orthogonality of the gene factor to the other factors effectively separates the effects into two groups: “global” or “non-gene” effects which involve A, D, and V, and gene-specific effects, such as the VG interaction, which involve G. The separation into global effects and gene-specific effects reduces the size of the matrix inversion by three to four or more orders of magnitude relative to the standard ANOVA methods.

The least-squares estimators, by definition, minimize the residual sum of squares (“RSS”) given by:

RSS=Σ(y_{ijkgs}−μ−Aᵢ−Dⱼ−Vₖ−G_{g}−(AG)_{igs}−(VG)_{kg}) (2)

where the sum is taken over all indices. The partial derivatives of the RSS with respect to each of the parameters gives the following set of linear equations.

$$\begin{matrix}\frac{δ RSS}{δμ} = 0 ⇒ {y}_{•••••} = \hat{μ} & ( 3 ) \\ \frac{δ RSS}{δ {A}_{i}} = 0 ⇒ {y}_{i ••••} = \hat{μ} + {\hat{A}}_{i} + \frac{1}{2} {\sum }_{k ∈ i} {\hat{V}}_{k} & ( 4 ) \\ \frac{δ RSS}{δ {D}_{{j}_{i}}} = 0 ⇒ {y}_{• j •••} = \hat{μ} + {\hat{D}}_{j} + \frac{2}{r} {\sum }_{k} {r}_{kj} {\hat{V}}_{k} & ( 5 ) \\ \frac{δ RSS}{δ {V}_{k}} = 0 ⇒ {y}_{•• k ••} = \hat{μ} + \frac{1}{{r}_{k}} {\sum }_{i ⊃ k} {\hat{A}}_{i} + \frac{{r}_{k1}}{{r}_{k}} {\hat{D}}_{1} + \frac{{r}_{k2}}{{r}_{k}} {\hat{D}}_{2} + {\hat{V}}_{k} & ( 6 ) \\ \frac{δ RSS}{δ {G}_{g}} = 0 ⇒ {y}_{••• g •} = \hat{μ} + {\hat{G}}_{g} & ( 7 )\end{matrix}$$

In the equations above, the “₅₀₅” as an index indicates an average over that index. For example, y_{•••••} is the average all the fluorescent intensity measurements in the experiment. Similarly, y_{1••••} is the average of all the fluorescent intensity measurements made on array 1. The “ˆ ” over a variable indicates the least-squares estimate for that variable. The notation k∈i means the varieties k appearing on array i such that if variety k appears on array i in both red and green channels, then it should appear twice in the summation. Similarly, i⊃k indicates a summation over the arrays i containing variety k such that if variety k appears on array i in both red and green channels, then it should appear twice in the summation. The equations above also incorporate the “zero-sum” constraints wherein ΣAᵢ=ΣDⱼ=ΣrₖVₖ=ΣG_{g}=Σ_{g}(VG)_{kg}=Σₖrₖ(VG)_{kg}=Σᵢₛ(AG)_{igs}=Σ_{gs}(AG)_{igs}=0 and rₖⱼ is the number of times variety k appears in the design labeled with dye j, rₖ is the total replication of variety k given by rₖ=Σ_{j=1,2}rₖⱼ, and r=Σrₖ.

Equations (4), (5), and (6) define a linear transformation, Tτ=d where

d^{T}={y_{1••••}, •••, y_{a-1••••}, y_{••1••}, •••, y_{••v-1••}, y_{•1•••}}−y_{•••••} (8)

τ^{T}={Â₁, •••, Âₐ₋₁, \hat{V}₁, •••, \hat{V}ᵥ₋₁, \hat{D}₁} (9)

where T is of size p′=a+v−1 which is much less than q. Since p′ is on the order of about 100, the matrix T may be inverted with commonly available matrix inversion algorithms. Alternatively, the system of equations, (4)-(6) may be solved directly by Q-R decomposition.

The two-factor least-squares estimators are given by equations (10) and (11) below.

$$\begin{matrix}{y}_{•• k g •} - {y}_{•• k ••} - {y}_{••• g •} + {y}_{•••••} = \frac{1}{{r}_{k}} {\sum }_{i ⊃ k} {( AG )}_{ig} + {( VG )}_{kg} & ( 10 ) \\ {y}_{i •• g •} - {y}_{i ••••} - {y}_{••• g •} + {y}_{•••••} = {( AG )}_{ig} + \frac{1}{2} {\sum }_{k ∈ i} {( VG )}_{kg} & ( 11 )\end{matrix}$$

Equations (10) and (11) define a linear transformation of the form T_{g}τ_{g}=d_{g} where

d^{T}_{g}={y_{1••g•}−y_{1••••}, •••, y_{a-1••g•}−y_{a-1••••}, y_{••1,g•}−y_{••1}_{••}, •••, y_{••v-1,g •}−y_{••v-1 ••}}−y_{•••g•}−y_{•••••} (12)

τ^{T}_{g}={(AG)_{1,g•}, •••, (AG)_{a-1,g•, (}VG)_{1,g}, •••, (VG)_{v-1,g}} (13)

and T_{g} is a square matrix of size a+v−2. T_{g} may be inverted with commonly available matrix inversion algorithms. More importantly, T_{g} is the same for every g and can be constructed from T. Alternatively, the system of equations, (10)-(11) may be solved directly by Q-R decomposition to obtain the two-factor estimates.

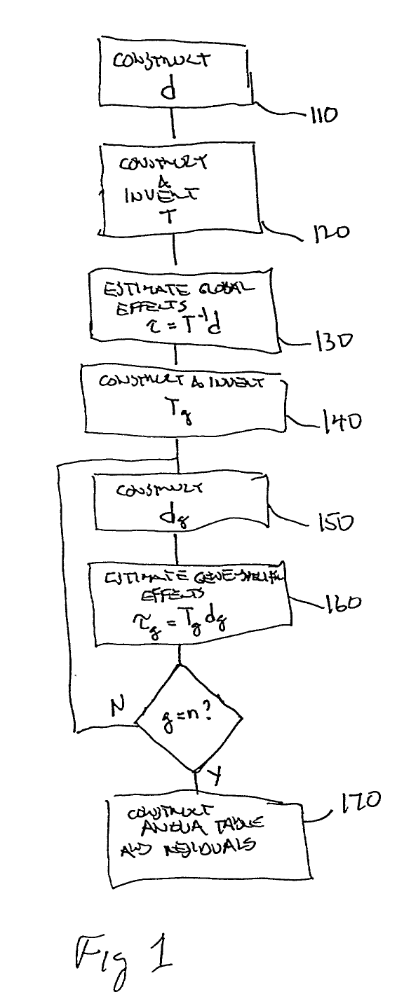

FIG. 1 shows a flowchart illustrating a computer implemented program of a preferred embodiment of the present invention. After the microarrays have been scanned and the fluorescent intensities measured and stored, the data vector, d, for the global factors is constructed in 110. The square matrix T is constructed and inverted in 120 using commonly available matrix inversion algorithms. The effects of the global factors are estimated in 130 by τ=T¹ d. The gene-specific matrix, T_{g}, is constructed and inverted in 140. For each gene in the experiment, the program first constructs the gene-specific data vector for the gth gene in 150 and estimates the two-factor interaction effects for the gth gene in 160. Steps 150 and 160 are repeated for each gene until all the gene-specific interactions have been estimated. Finally, the program calculates the ANOVA table and residuals in 170 based on the estimates using standard techniques known to one of ordinary skill in the statistical art.

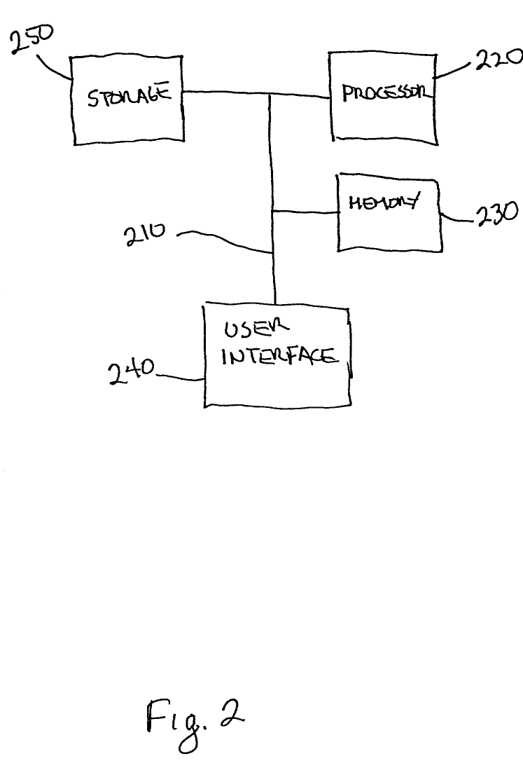

FIG. 2 shows a block diagram of another embodiment of the present invention. A bus 210 is connected to a processor 220 and provides signal communication between the processor 220 and memory 230, user interface 240, and storage 250. The user interface 240 allows the processor to display, print or send information to the user and receive data input from the user or another external source. Storage 250 is capable of permanently storing data and programs executable by the processor that may be used by the processor 220. Data and programs may be transferred to memory 230 for faster access by the processor 220. In a preferred embodiment of the present invention, a program embodiment of the flowchart of FIG. 1 is stored in storage 250. A user may command the processor 220 to execute the program via the user interface 240. The processor 220 may also receive a dataset via the user interface 240 or the dataset may have been previously stored in storage 250. The processor 220 executes the program and uses the dataset to calculate the ANOVA tables and residuals. After the processor 220 has calculated the tables and residuals, the processor 220 may either display or print the tables and residuals for the user's review or may store the tables and residuals in storage 250.

It should be apparent to one of ordinary skill in the statistical modeling art that the present invention is not limited by the choice of the data model described in equation (1). For example, another embodiment of the present invention includes a data model that replaces the variety effect with the array-dye interaction. In addition, the dye-gene interaction may also be added to the data model.

In another embodiment of the present invention, two data models are used to analyze the dataset. The first data model includes only non-gene factors and interactions. An example of such a data model is given by the equation below.

y_{ijkgs}=μ+Aᵢ+Dⱼ+(AD)ᵢⱼ+ε_{ijkgs} (14)

The A, D, and AD effects may be estimated independent of the gene-specific factors because of the orthogonality of the gene factor to the other non-gene factors. Using the estimates of the A, D, and AD effects obtained using the data model of equation (14), the dataset is transformed using the equation below.

x_{ijkgs}=y_{ijkgs}−\hat{μ}−Âᵢ−\hat{D}ⱼ−(AD)ᵢⱼ (15)

A second data model including only the gene-specific factors is constructed for the transformed dataset having the form shown in the equation below.

x_{ijkgs}=\hat{μ}_{g}+(AG)_{igs}+(DG)_{jg+}(VG)_{kg} (16)

The gene-specific effects may be estimated using the second data model gene by gene and therefore does not require simultaneously solving for all gene effects at once.

The present invention is not to be limited in scope by the specific embodiments described herein. Indeed, modifications of the invention in addition to those described herein will become apparent to those skilled in the art from the foregoing description and accompanying figures. Doubtless, numerous other embodiments can be conceived that would not depart from the teaching of the present invention, whose scope is defined by the following claims.

We claim:

1. A method for estimating the effects of a plurality of factors and at least one of a plurality of interactions between the factors in a gene expression microarray experiment generating a microarray dataset wherein the factors include a gene factor and at least one non-gene factor and the interactions include at least one gene interaction, the gene factor being orthogonal to the other factors, the method comprising the steps of:
(a) estimating the factor effects based on a plurality of averages of the microarray dataset; and
(b) estimating the at least one gene interaction effects based on a plurality of averages of the microarray dataset and on the estimated factor effects from step (a).

2. The method of claim 1 wherein each non-gene factor is characterized by a number of levels, the step of estimating the main effects includes inverting a square matrix of size p wherein p is equal to the sum of the number of levels for each non-gene factor minus the number of non-gene factors.

3. The method of claim 1 wherein each factor is characterized by a level, the step of estimating the interaction effects includes inverting a square matrix of size p′ wherein p′ is equal to the sum of the number of levels for each non-gene factor minus the number of non-gene factors minus one.

4. The method of claim 3 wherein the non-gene factor includes a variety factor and the step of estimating the interaction effects further includes estimating the variety-gene interaction effects for each gene based on the inverted square matrix of size p′.

5. The method of claim 1 wherein the non-gene factors includes a variety factor.

6. The method of claim 1 wherein the non-gene factors includes an array factor.

7. The method of claim 1 wherein the non-gene factors includes a dye factor.

8. The method of claim 7 wherein the dye factor has two levels.

9. The method of claim 6 wherein the array factor has two levels.

10. The method of claim 8 wherein the array factor is balanced with respect to the dye factor.

11. A method for estimating at least one gene-variety interaction in a gene expression microarray experiment having an experimental design characterized by a number of degrees of freedom, q, and defined by a gene factor, a plurality of non-gene factors, a plurality of two-factor interactions wherein a full replication of genes is present for every combination of the plurality of non-gene factors, the method comprising the steps of:
(a) inverting a first square matrix characterized by a size, p, wherein p<q;
(b) estimating at least one of a plurality of non-gene factor effect from the first square matrix inverse;
(c) constructing a second square matrix based in part on the estimated non-gene factor, the second square matrix characterized by size, p′, wherein p′<q;
(d) inverting a second square matrix; and
(e) estimating at least one gene-variety interaction from the inverted second square matrix.

12. A method for estimating at least one gene-variety interaction in a gene expression microarray experiment generating a dataset and having a design characterized by a arrays, v varieties, n genes, and d dyes wherein a full replication of genes is present for every combination of arrays, varieties and dyes, the method comprising the steps of:
(a) constructing a global data vector, d, based on a plurality of averages of the dataset;
(b) constructing a square matrix, T, characterized by a size, p, wherein p=a+v+d−3;
(c) inverting the square matrix, T;
(d) estimating the global effects, τ, wherein τ=T d;
(e) constructing a square matrix, T_{g}, characterized by a size, p′, wherein p′=p−1;
(f) constructing a gene-specific data vector, d_{g}, based on a plurality of averages of the dataset;
(g) inverting the square matrix, T_{g}; and
(g) estimating the gene-variety interaction, τ_{g}, wherein τ_{g}=T_{g}d_{g}.

13. A system for estimating the effects of a plurality of factors and at least one of a plurality of interactions between the factors in a gene expression microarray experiment generating a microarray dataset wherein the factors include a gene factor and a variety factor and the interactions include a variety-gene interaction, the gene factor being orthogonal to the other factors, the system comprising:
(a) a processor;
(b) a memory in signal communication with the processor;
(c) a program stored in the memory, the program capable of being executed by the processor, the program including the steps of:
(i) estimating the main effects based on a plurality of averages of the microarray dataset; and
(ii) estimating the interaction effects based on a plurality of averages of the microarray dataset and on the estimated factor effects from step (i).

14. The system of claim 13 wherein each factor is characterized by a level, the step of estimating the main effects includes inverting a square matrix of size p wherein p is equal to the sum of the levels for each non-gene factor minus the number of non-gene factors.

15. The system of claim 13 wherein each factor is characterized by a level, the step of estimating the interaction effects includes inverting a square matrix of size p′ wherein p′ is equal to the sum of the levels for each non-gene factor minus the number of non-gene factors minus one.

16. The system of claim 15 wherein the step of estimating the interaction effects further includes estimating the variety-gene interaction effects for each gene based on the inverted square matrix of size p′.

17. The system of claim 13 wherein the non-gene factors includes a variety factor.

18. The system of claim 13 wherein the non-gene factors includes an array factor.

19. The system of claim 13 wherein the non-gene factors includes a dye factor.

20. The system of claim 19 wherein the dye factor has two levels.

21. The system of claim 18 wherein the array factor has two levels.

22. The system of claim 20 wherein the array factor is balanced with respect to the dye factor.

23. The method of claim 1 wherein the interactions further include an array-dye interaction.

24. The system of claim 13 wherein the interactions further include an array-dye interaction.

25. A method for estimating the effects of a plurality of factors and at least one of a plurality of interactions between the factors in a gene expression microarray experiment generating a microarray dataset wherein the factors include a gene factor and a plurality of non-gene factors and the interactions include at least one of a gene-non-gene interaction, the gene factor being orthogonal to the non-gene factors, the method comprising the steps of:
(a) constructing a first data model including only non-gene factors and non-gene interactions;
(b) estimating the effects of the non-gene factors and non-gene interactions based on the first data model and on a plurality of averages of the microarray dataset;
(b) creating a transformed dataset from the microarray dataset and the factor and interaction effects estimated in step (a);
(c) constructing a second data model including the gene factors and the gene interactions; and
(d) estimating the gene-non-gene interaction effects based on the second data model and a plurality of averages of the transformed dataset.

26. The method of claim 25 wherein the non-gene factors includes a variety factor.

27. The method of claim 26 wherein the non-gene factors includes an array factor.

28. The method of claim 27 wherein the non-gene factors includes a dye factor.

29. The method of claim 28 wherein the dye factor has two levels.

30. The method of claim 27 wherein the array factor has two levels.

31. The method of claim 30 wherein the array factor is balanced with respect to the dye factor.

32. The method of claim 28 wherein a non-gene interaction includes an array-dye interaction.

33. The method of claim 32 wherein the transformed dataset is created according to the equation:
x_{ijkgs}=y_{ijkgs}−\hat{μ}−Âᵢ−\hat{D}ⱼ−(AD)ᵢⱼ
where
x_{ijkgs} is the transformed measurement of y_{ijkg} measurement,
y_{ijkg} is the ijkgs^{th} measurement;
\hat{μ} is the estimated mean of all measurements;
Âᵢ is the estimated array effect for the i^{th} array;
\hat{D}ⱼ is the estimated dye effect for the j^{th} dye;
(AD)ᵢⱼ is the estimated array-dye interaction effect of the i^{th} array and the j^{th} dye.

34. A computer readable media comprising instructions encoded therein for a computer to perform the method of claim 1.
